(19) United States
(12) Patent Application Publication
(10) Pub. No.: US 2024/0350424 A1
(43) Pub. Date: Oct. 24, 2024

(54) TRANSDERMAL PATCHES FOR ADMINISTERING COMPOUNDS AND BODY ADORNMENT

(71) Applicant: Barriere, Inc., Brooklyn, NY (US)

(72) Inventors: Alexa Adams, Brooklyn, NY (US); Cleo Davis-Urman, Brooklyn, NY (US)

(21) Appl. No.: 18/641,871

(22) Filed: Apr. 22, 2024

Related U.S. Application Data
(60) Provisional application No. 63/461,127, filed on Apr. 21, 2023.

(51) Int. Cl. A61K 9/70; A61F 13/0246; A61M 37/00

(52) U.S. Cl. CPC A61K 9/7038; A61F 13/0246; A61M 37/00

(57) ABSTRACT

Transdermal patches including a substrate, an adhesive, and a compound formulation. The substrate is configured as a planer member with a first major face and a second major face opposite the first major face. The substrate is configured to mount to skin and includes a bottom side and a top side. The bottom side defines the first major face of the substrate and faces the skin when the substrate is mounted to the skin. The top side is opposite the bottom side and defines the second major face of the substrate. The top side includes an ornamental design. The adhesive is disposed on the bottom side of the substrate and is configured to selectively couple the substrate to the skin. The compound formulation is disposed on the bottom side of the substrate and is configured to absorb through the skin into a bloodstream below the skin.

### CROSS REFERENCE TO RELATED APPLICATIONS

This application claims priority to copending U.S. Application, Ser. No. 63/461,127, filed on Apr. 21, 2023, which is hereby incorporated by reference for all purposes.

### BACKGROUND

The present disclosure relates generally to transdermal patches. In particular, transdermal patches for administering compounds and adorning one's body are described.

A variety of compounds are beneficial or desirable for humans. Vitamins, supplements, adaptogens, and pharmaceuticals are examples of beneficial compounds. Increasingly, compounds like cannabis and psychedelics are being legalized and considered as beneficial for certain medical conditions. Some people also wish to experience the effects of tobacco.

Adorning one's body is an important means of self-expression. Known methods for adorning one's body include permanent tattooing, temporary tattooing, painting, wearing jewelry, or applying stickers. Methods like tattooing, painting, or wearing jewelry can be undesirably permanent, time consuming, and/or expensive.

Compounds can be administered to people in various ways. For example, compounds may be administered by injecting them, ingesting them, inhaling them, or absorbing them through the skin, that is, transdermally. Conventional methods for administering compounds have various drawbacks.

For example, injections are not pleasant or tolerable for certain people. Ingesting selected compounds is not always practical or convenient. Further, ingestion requires that the selected compound be incorporated into food or other edible vector, which often tastes unpleasant or has a limited shelf life. Inhaling compounds often requires smoking, which carries social stigma, is not allowed in many places, and may be painful or unpleasant for certain people.

Patches exist to administer compounds transdermally, but have limitations. Conventional patches lack expressive designs or are designed to have any image or logo present on them fade away to be less visually apparent. Rather than provide an enduring form of self-expression via body adornment, conventional transdermal patches are typically intended to be invisible or discreet.

Certain conventional transdermal patches have clear substrates, but do not enable designs to be printed on them. An inability to print designs on the clear substrate limits their utility for adornment and expression. Further, conventional transdermal patches with clear substrates do not appear as tattoos given their lack of printed designs.

Conventional transdermal patches that accommodate artwork to some extent, such as adhesive bandages, e.g., Band-Aid® adhesive bandages, are often bulky and are not clear. Instead, thick fabric substrates with opaque coloring is typically necessary for conventional transdermal patches to display artwork.

Thus, there exists a need for means to administer selected compounds that improve upon and advance the design of known means. Examples of new and useful transdermal patches for administering compounds and adorning one's body relevant to the needs existing in the field are discussed below.

### SUMMARY

The present disclosure is directed to transdermal patches including a substrate, an adhesive, and a compound formulation. The substrate is configured as a planer member with a first major face and a second major face opposite the first major face. The substrate is configured to mount to skin and includes a bottom side and a top side.

The bottom side defines the first major face of the substrate and faces the skin when the substrate is mounted to the skin. The top side is opposite the bottom side and defines the second major face of the substrate. The top side includes an ornamental design.

The adhesive is disposed on the bottom side of the substrate and is configured to selectively couple the substrate to the skin. The compound formulation is disposed on the bottom side of the substrate and is configured to absorb through the skin into a bloodstream below the skin.

### BRIEF DESCRIPTION OF THE DRAWINGS

FIG. 1 is a plan view of a set of transdermal patches for administering compounds with a first set of ornamental designs.

FIG. 2 is a plan view of a set of transdermal patches for administering compounds with a second set of ornamental designs.

FIG. 3 is a plan view of a set of transdermal patches for administering compounds with a third set of ornamental designs.

FIG. 4 is a view of a transdermal patch from the set shown in FIG. 2 adhered to an arm.

FIG. 5 is a bottom view of a transdermal patch shown in FIG. 4 with a compound formulation mixed into an adhesive applied as a combined layer to the bottom of a substrate.

FIG. 6 is a side elevation view of a portion of the transdermal patch shown in FIG. 4 depicting the compound formulation and the adhesive defining a combined layer on the bottom of the substrate.

FIG. 7 is a side elevation view of a portion of an alternative transdermal patch depicting a compound formulation layer disposed on top of an adhesive layer, which is applied to a bottom of a substrate.

FIG. 8 is a perspective view of an additional example of a transdermal patch above a person's skin with layers of the transdermal patch separated from each other.

### DETAILED DESCRIPTION

The disclosed transdermal patches will become better understood through review of the following detailed description in conjunction with the figures. The detailed description and figures provide merely examples of the various inventions described herein. Those skilled in the art will understand that the disclosed examples may be varied, modified, and altered without departing from the scope of the inventions described herein. Many variations are contemplated for different applications and design considerations; however, for the sake of brevity, each and every contemplated variation is not individually described in the following detailed description.

Throughout the following detailed description, examples of various transdermal patches are provided. Related features in the examples may be identical, similar, or dissimilar in different examples. For the sake of brevity, related features will not be redundantly explained in each example. Instead, the use of related feature names will cue the reader that the feature with a related feature name may be similar to the related feature in an example explained previously. Features specific to a given example will be described in that particular example. The reader should understand that a given feature need not be the same or similar to the specific portrayal of a related feature in any given figure or example.

### Definitions

The following definitions apply herein, unless otherwise indicated.

“Substantially” means to be more-or-less conforming to the particular dimension, range, shape, concept, or other aspect modified by the term, such that a feature or component need not conform exactly. For example, a “substantially cylindrical” object means that the object resembles a cylinder, but may have one or more deviations from a true cylinder.

“Comprising,” “including,” and “having” (and conjugations thereof) are used interchangeably to mean including but not necessarily limited to, and are open-ended terms not intended to exclude additional elements or method steps not expressly recited.

Terms such as “first”, “second”, and “third” are used to distinguish or identify various members of a group, or the like, and are not intended to denote a serial, chronological, or numerical limitation.

“Coupled” means connected, either permanently or releasably, whether directly or indirectly through intervening components.

### Transdermal Patches for Administering Compounds and Body Adornment

With reference to the figures, transdermal patches for administering compounds and body adornment will now be described. The transdermal patches discussed herein function to administer selected compounds through the skin of the person wearing the skin patch. Further, the transdermal patches provide a way for the wearer to adorn his or her body with a selected ornamental design. The transdermal patches described herein mimic tattoos.

The reader will appreciate from the figures and description below that the presently disclosed transdermal patches address many of the shortcomings of conventional means for administering compounds. For example, the novel transdermal patches described below allow for administering compounds without the attendant drawbacks of injections, ingestion, or inhalation. Further, the novel transdermal patches enable one to adorn his or her body with a selected ornamental design without the permanence, time consumption, or expense of many conventional methods of body adornment.

Unlike conventional transdermal patches, which are intended to be invisible or discreet, the novel transdermal patches described below have bright, vivid, multi-color designs to stand out. Rather than having images or logos configured to fade away to be less visually apparent like with conventional transdermal patches, the novel transdermal patches have enduring, expressive designs.

A notable improvement over conventional transdermal patches is that the novel transdermal patches discussed herein have clear substrates, yet enable designs to be printed on them. Accordingly, the novel transdermal patches provide adornment and expression utility not present in conventional transdermal patches. Further, the novel transdermal patches can advantageously be configured to appear as tattoos with their printed designs on clear substrates.

Beneficially, the novel transdermal patches discussed below accommodate artwork without requiring bulky and opaque substrates. Instead, the novel transdermal patches can accommodate artwork with thin and clear substrates, which enable the substrates to appear substantially invisible except for the design displayed on them.

### Transdermal Patch Embodiment One

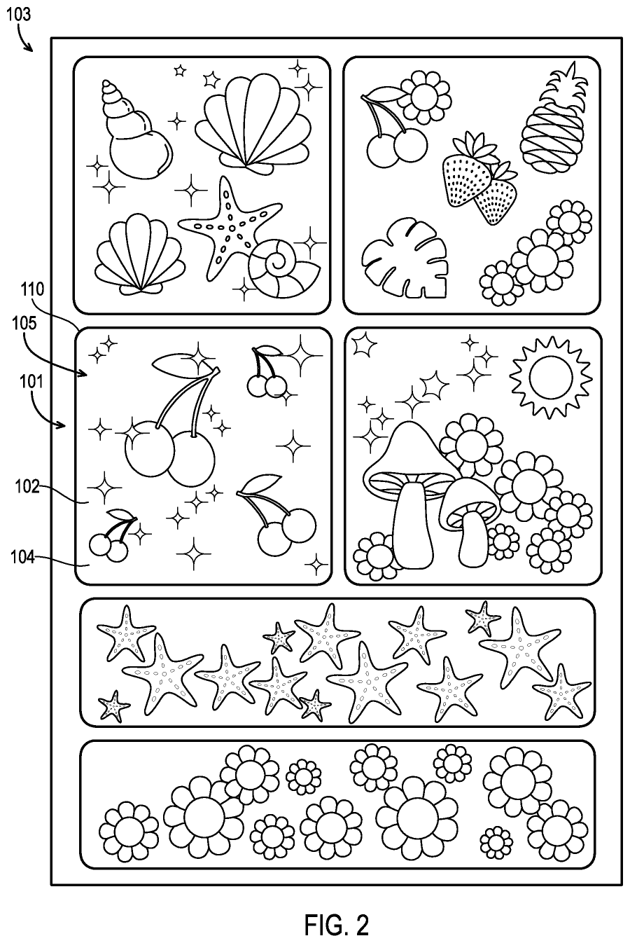

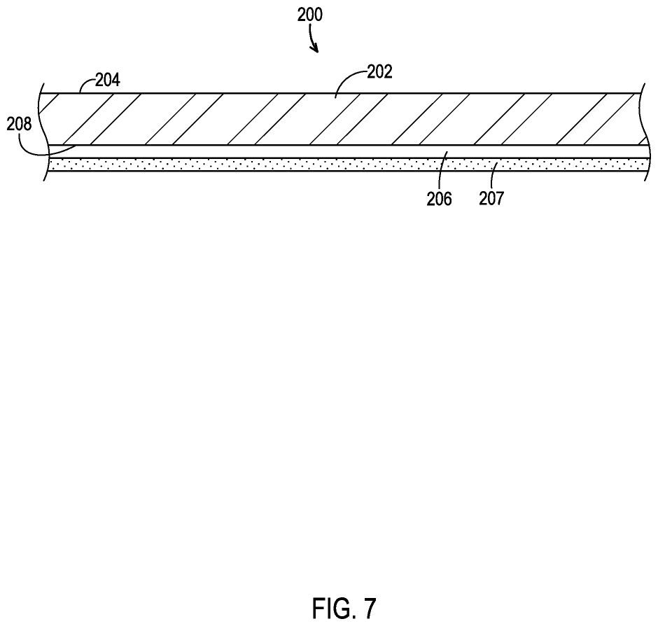

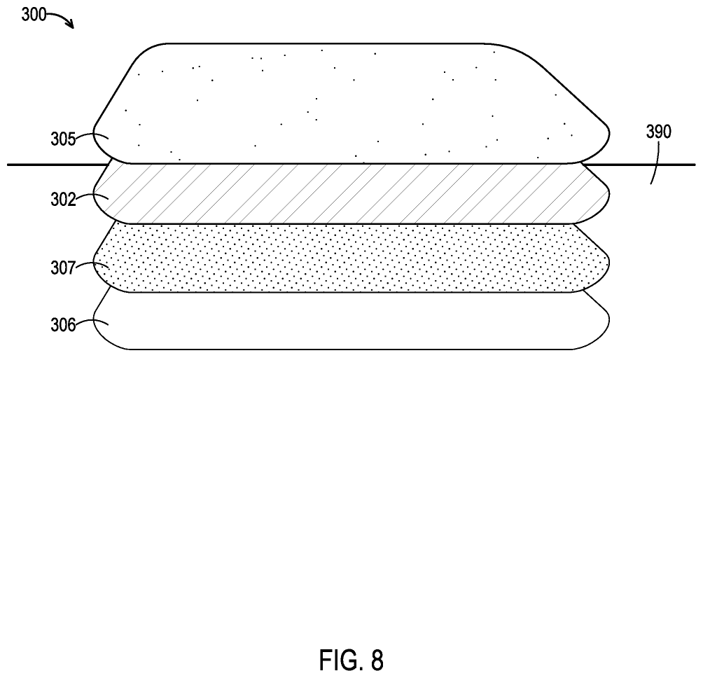

With reference to FIGS. 2 and 4-6, a first example of a transdermal patch, transdermal patch 100, will now be described. A second example of a transdermal patch, transdermal patch 200, is depicted in FIG. 7 and discussed further below. A third example of a transdermal patch, transdermal patch 300, is depicted in FIG. 8 and discussed further below as well.

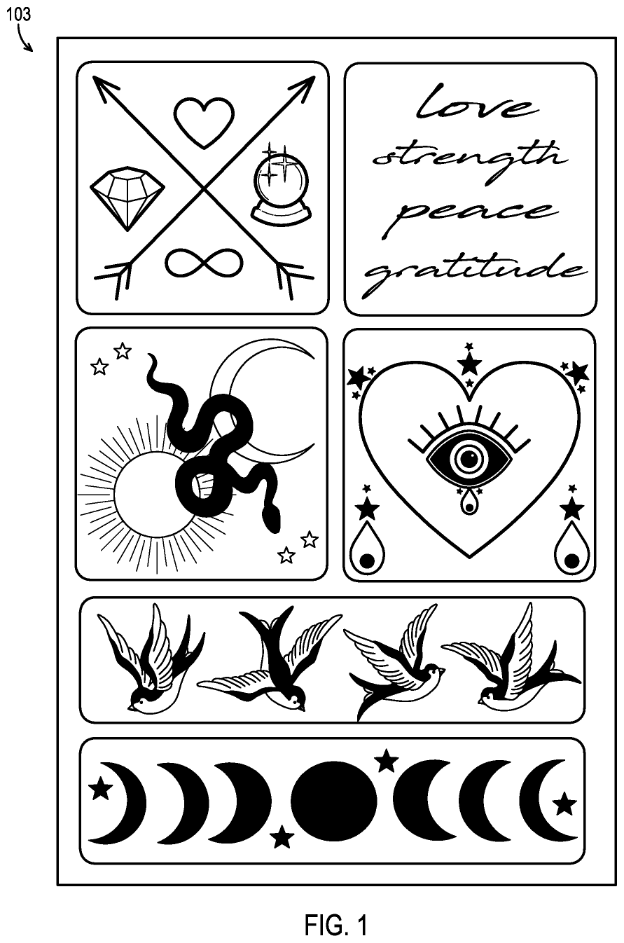

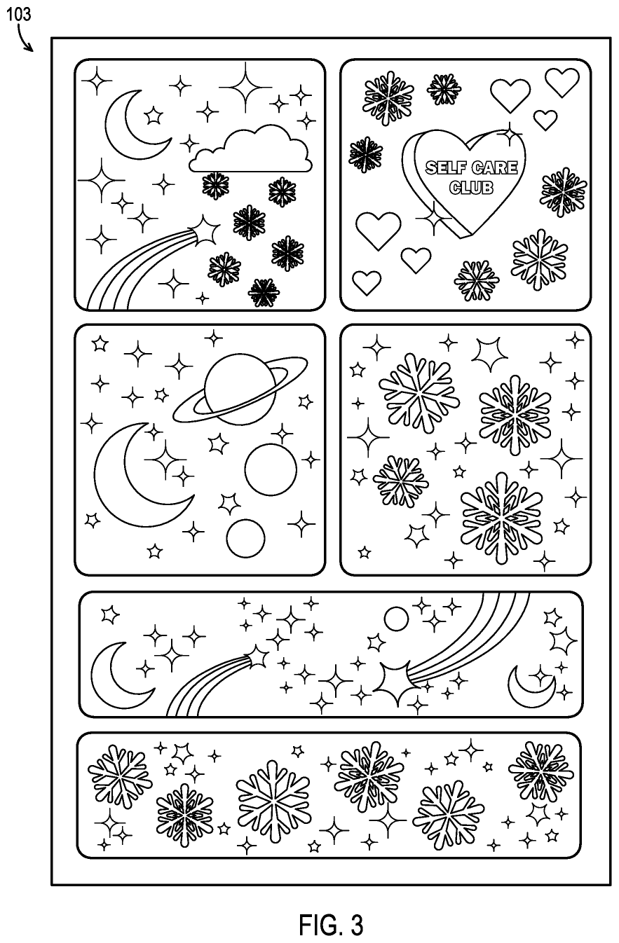

A variety of transdermal patch examples are shown in FIGS. 1-3. The various transdermal patches depicted in FIGS. 1-3 are configured the same except in size, shape, and ornamental design displayed.

Transdermal patch 100 includes a substrate 102, an adhesive layer 106, a compound formulation 107, and a backing sheet 101. In some examples, the transdermal patch does not include one or more features included in transdermal patch 100. In other examples, the transdermal patch includes additional or alternative features. The components of transdermal patch 100 are discussed further below.

FIGS. 1-3 depict transdermal patches organized into transdermal patch sheets 103. The reader should understand, however, that the transdermal patches described in this document may be individually packaged and/or not organized into a set or sheet.

In the examples shown in FIGS. 1-3, each transdermal patch sheet 103 includes six transdermal patches. Alternatively, the transdermal patches may be organized into sheets with more or fewer transdermal patches than the six patch sheets depicted in the figures, such as two patch sets, three patch set, eight patch sets, or sets of ten or more patches. The discussion below will focus on an individual transdermal patch unless reference is made to transdermal patch sheets.

The reader can see in FIGS. 1-3 that the transdermal patches on a given sheet may vary in size, shape, and design. For example, in FIG. 1, four of the transdermal patches are square and two are rectangular. The width of the rectangle shaped patches is approximately double the width of the square patches. The height of the rectangle shaped patches is approximately half the height of the square patches.

As a result of the width and height relationship between the different shapes, the surface area of the bottoms of the square and rectangular patches is approximately the same despite being different shapes. Having an approximately equal surface area is beneficial for delivering an equal dose of the compound formulation.

In FIGS. 1-3, the transdermal patches of each transdermal patch sheet all have different ornamental designs. However, the ornamental designs of the transdermal patches in the transdermal patch sheets are related by having a common theme. In some examples, the transdermal patches in a given sheet have unrelated ornamental designs. In certain examples, the ornamental designs of all the patches in a sheet are the same.

### Substrate

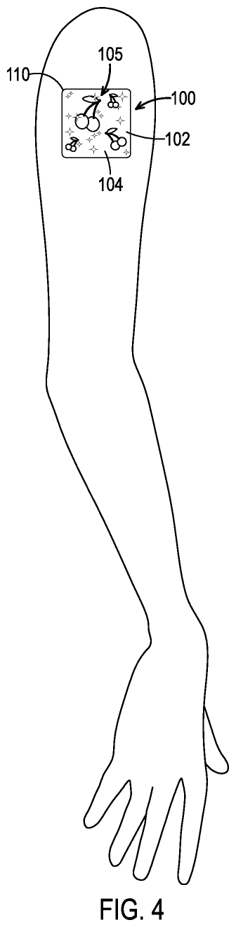

Substrate 102 functions to support adhesive layer 106 and compound formulation 107. Substrate 102 also enables compound formulation 107 to be delivered to a person's skin. As shown in FIG. 4, substrate 102 further enables one to adorn his or her body by displaying an ornamental design 105 and cooperating with adhesive layer 106 to selectively mount to a person's skin.

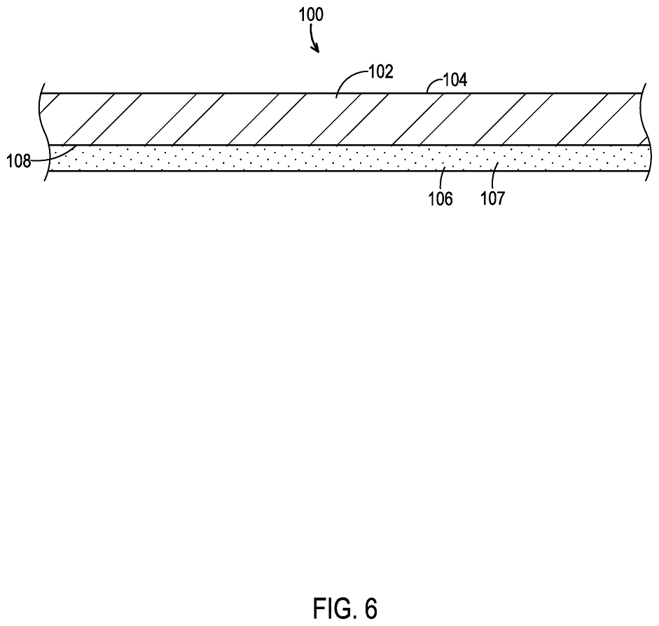

With reference to FIG. 6, the reader can see that substrate 102 is configured as a planer member with a first major face and a second major face. The second major face is disposed opposite the first major face. The distance between the major faces of substrate 102 is defined as a thickness of substrate 102.

The thickness of substrate 102 between the first major face and the second major face is selected to make substrate less visually apparent. In the present example, substrate 102 is 80 microns thick. In other examples, the substrate is less than 80 microns thick. It has been observed that the substrate having a thickness of 80 microns or less is critical to make the substrate visually unapparent or substantially invisible. However, in some examples where the substrate being visually apparent is acceptable or desired, the substrate is thicker than 80 microns. The thickness of the substrate may be selected to provide a desired amount of structural integrity and visual apparentness.

The composition of substrate 102 is also selected to make it less visually apparent and nearly invisible. In the present example, substrate 102 is composed of polyethylene terephthalate, which is also referred to as PET plastic. Through experimentation, the inventors have discovered that PET plastic is uniquely well suited to making substrate 102 appear invisible. A wide range of other substrate compositions are suitable, but PET plastic is preferred for purposes of making substrate 102 less visually apparent or substantially invisible.

As further shown in FIG. 6, substrate 102 includes a bottom side 108 and a top side 104. Top side 104 is opposite bottom side 108. As shown in FIG. 2, substrate 102 is removably mounted to backing sheet 101. Bottom side 108 faces backing sheet 101 when substrate 102 is selectively mounted to backing sheet 101 via adhesion from adhesive layer 106.

Bottom side 108 defines the first major face and top side 104 defines the second major face. As is apparent in FIG. 4, bottom side 108 faces the skin of a wearer when substrate 102 is mounted to the skin. Ornamental design 105 is displayed on top side 104.

As shown in FIG. 4, substrate 102 is configured to appear as a tattoo when worn on the body. In FIG. 4, the edges of substrate 102 are exaggerated and more visually apparent than they would be in reality to demonstrate the size, shape, and function of transdermal patch 100. In actual use, substrate 102 is thin and clear with unapparent edges to make it nearly invisible except for ornamental design 105 displayed on top side 104. In other examples, the substrate is configured as a sticker and is more visually apparent than substrate 102.

The shape of the substrate may be selected to provide a desired aesthetic appearance or functional attribute. For example, as depicted in FIGS. 2, 4, and 5, substrate 102 has rounded corners 110 to be more aesthetically pleasing and to minimize sharp edges that may be more visually noticeable on a person's skin. In other examples, the shape of the substrate is rectangular, oval, circular, a regular polygon, irregular, or a shape conforming to the shape of the ornamental design displayed on the substrate.

As shown in FIGS. 2 and 4, ornamental design 105 is displayed on top side 104 of substrate 102. The reader can see in FIGS. 1-4 that the ornamental design may be any manner of design, including objects, plants, animals, celestial bodies, text, and fanciful subjects. A wide variety of other designs are contemplated. The ornamental design may be any design one might select for a tattoo or any other design that can be printed or applied to top side 104 of substrate 102.

As is apparent from FIGS. 1-3, the ornamental designs of the patches on a given sheet may share a common theme. However, the ornamental designs of different patches on a given sheet may be unrelated or may be identical.

The ornamental designs may be in black and white colors. Additionally or alternatively, the ornamental designs may be in a wide range of colors beyond black and white as well as patterns and color gradients.

In the present example, ornamental design 105 is applied to top side 104 by a digital printing machine. However, the ornamental design may be applied to the substrate by any currently known or later developed means.

### Adhesive Layer

As shown in FIG. 4, adhesive layer 106 functions to selectively adhere substrate 102 to a person's skin. Adhesive layer 106 also serves to selectively secure substrate 102 to backing sheet 101 until one is ready to use transdermal patch 100 to administer compound formulation 107 transdermally.

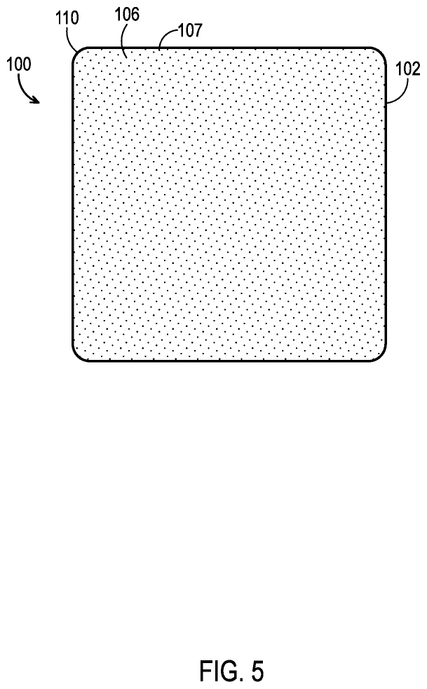

As shown in FIGS. 5 and 6, adhesive layer 106 is applied to a bottom side 108 of substrate 102. Bottom side 108 faces backing sheet 101 and is opposite top side 104 of substrate 102 on which ornamental design 105 is displayed. The adhesive layer may be applied by any currently known or later developed means, including spraying, dispensing, or pressing adhesive onto bottom side 108 of substrate 102.

The adhesive layer may be any adhesive formulation currently known or later developed suitable for adhering substrates to a person's skin and to backing sheet 101. In the present example, adhesive layer 106 is formulated to removably adhere substrate 102 to backing sheet 101 and to the skin. Further, adhesive formulation 106 is formulated to have a desired length of adherence to a user's skin and to require a selected amount of force to separate substrate 102 from a person's skin. In the example shown in FIGS. 2 and 4-6, adhesive formulation 106 is selected to be clear in color to enhance the tattoo-like appearance of ornamental design 105 displayed on substrate 102.

As depicted in FIGS. 5 and 6, compound formulation 107 is mixed with adhesive layer 106 in transdermal patch 100. In contrast, in the transdermal patch 200 example depicted in FIG. 7, adhesive layer 206 and compound formulation 207 are separate layers rather than intermixed together. In particular, adhesive layer 206 is disposed between compound formulation 207 and bottom side 208 of substrate 202 opposite top side 204. The size of the layers depicted in the FIGS. 6 and 7 section views are not to scale.

A further configuration of a transdermal patch 300 is depicted in FIG. 8. As shown in FIG. 8, transdermal patch 300 includes a printed ornamental design 305 oriented on top of a substrate 302. Below substrate 302 is first a compound formulation layer 307 and then an adhesive layer 306 disposed between compound formulation layer 307 and a person's skin 390. The reader will note that the adhesive layer and the compound formulation layer are in opposite positions relative to the substrate in FIG. 8 than they are in FIG. 7, which demonstrates that the different layer orientations are suitable in different examples.

The amount and thickness of the adhesive layer may be selected to meet the needs of a given application. In instances where the adhesive formulation has stronger adhesive properties or less adhesion is desired, a smaller amount of the adhesive and/or a thinner layer of the adhesive may be applied to the substrate. In instances where the adhesive formulation has relatively weak adhesive properties or more adhesion is desired, a larger amount of the adhesive and/or a thicker layer of the adhesive may be applied to the substrate.

### Compound Formulation

The compound formulation functions to impart health promoting or other desired effects to a person when absorbed through the person's skin into the person's bloodstream. The compound formulation may enhance a person's wellbeing and/or address health issues one is facing, such as an illness or ailment. The compound formulation may also improve one's mood or help maintain or improve one's mental health. In some examples, the compound formulation is formulated for recreational purposes, such as to provide narcotic, psychoactive, or inebriation effects.

Compound formulation 107 is configured to absorb through the skin into a bloodstream below the skin. The compound formulation may be any currently known or later developed formulation of compounds suitable to be administered to humans or animals through the skin. In some examples, the formulation is a single compound. In other examples, the formulation is mixture of two or more different compounds.

The compounds may include one or more of vitamins, supplements, adaptogens, pharmaceuticals, and therapeutic compounds. Additionally or alternatively, the compounds may include one or more of cannabinoids, psychedelics, psychoactive compounds, narcotics, and tobacco. The selection of compounds and the ratios of compounds may vary widely and be selected to achieve any desired effect in people.

As shown in FIGS. 5 and 6, compound formulation 107 is mixed with adhesive layer 106 and the mixture is applied to bottom side 108 of substrate 102. The compound formulation mixed with the adhesive defines an adhesive-compound formulation mixture. In the FIG. 7 transdermal patch 200 example, compound formulation 207 is applied as a separate layer onto adhesive layer 206, which is disposed on bottom side 208 of substrate 202. In the alternative configuration depicted in FIG. 8, adhesive layer 306 is disposed onto a compound formulation layer 307, which is disposed on the bottom of substrate 302.

The compound formulation may be applied to the substrate or to the adhesive layer by any currently known or later developed means. Suitable application means include spraying, dispensing, or pressing the compound formulation onto the bottom side of the substrate and/or onto the adhesive layer.

### Backing Sheet

As can be seen in FIGS. 1-3, backing sheet 101 functions to support substrate 102. Further, backing sheet 101 serves to cover adhesive layer 106 and compound formulation 107. Covering adhesive layer 106 and compound formulation 107 inhibits them degrading from exposure to air. Further, covering adhesive layer 106 helps avoid substrate 102 adhering to an unintended object.

In the examples shown in FIGS. 2 and 4, backing sheet 101 is a clear Mylar sheet. However, the backing sheet may be comprised of any suitable material for backing applications, such as other polymers, metals, and composite materials. The backing sheet may be any currently known or later developed type of backing sheet.

In examples where the transdermal patch is organized into sets or sheets, such as shown in FIGS. 1-3, backing sheet 101 will be sized to accommodate all the substrates in the set. When the transdermal patch is individually packaged or presented, the backing sheet will be complementarily configured with the substrate to be somewhat larger than the substrate. Backing sheet 101 being larger than substrate 102 facilitates selectively removing substrate 102 from backing sheet 101.

In examples where transdermal patch is organized into sets or sheets, the backing sheet may be scored between the substrates to facilitate selectively separating a substrate from the set. However, scoring is optional and backing sheet 101 shown in the figures is not scored.

The disclosure above encompasses multiple distinct inventions with independent utility. While each of these inventions has been disclosed in a particular form, the specific embodiments disclosed and illustrated above are not to be considered in a limiting sense as numerous variations are possible. The subject matter of the inventions includes all novel and non-obvious combinations and subcombinations of the various elements, features, functions and/or properties disclosed above and inherent to those skilled in the art pertaining to such inventions. Where the disclosure or subsequently filed claims recite “a” element, “a first” element, or any such equivalent term, the disclosure or claims should be understood to incorporate one or more such elements, neither requiring nor excluding two or more such elements.

Applicant(s) reserves the right to submit claims directed to combinations and subcombinations of the disclosed inventions that are believed to be novel and non-obvious. Inventions embodied in other combinations and subcombinations of features, functions, elements and/or properties may be claimed through amendment of those claims or presentation of new claims in the present application or in a related application. Such amended or new claims, whether they are directed to the same invention or a different invention and whether they are different, broader, narrower or equal in scope to the original claims, are to be considered within the subject matter of the inventions described herein.

1. A transdermal patch, comprising:
a substrate configured as a planer member with a first major face and a second major face opposite the first major face, the substrate configured to mount to skin, the substrate including:
a bottom side defining the first major face of the substrate, the bottom side facing the skin when the substrate is mounted to the skin; and
a top side opposite the bottom side and defining the second major face of the substrate, the top side including an ornamental design;
an adhesive disposed on the bottom side of the substrate, the adhesive configured to selectively couple the substrate to the skin; and
a compound formulation operatively disposed on the bottom side of the substrate, the compound formulation configured to absorb through the skin into a bloodstream below the skin.

2. The transdermal patch of claim 1, wherein the compound formulation is mixed with the adhesive layer.

3. The transdermal patch of claim 1, wherein the adhesive layer is disposed between the compound formulation and the bottom side of the substrate.

4. The transdermal patch of claim 1, wherein the substrate has a thickness selected to make the substrate visually unapparent when the substrate is mounted to the skin.

5. The transdermal patch of claim 4, wherein the substrate has rounded corners.

6. The transdermal patch of claim 4, wherein the substrate has a thickness of 80 microns or less.

7. The transdermal patch of claim 6, wherein the ornamental design is printed onto the top side of the substrate with a digital printer.

8. The transdermal patch of claim 6, wherein the substrate has rounded corners.

9. The transdermal patch of claim 1, wherein the substrate appears to be a tattoo in the form of the ornamental design when the substrate is mounted to the skin.

10. The transdermal patch of claim 1, wherein the compound formulation includes a therapeutic compound.

11. The transdermal patch of claim 1, wherein the compound formulation includes a narcotic compound.

12. The transdermal patch of claim 1, wherein the compound formulation includes a psychoactive compound.

13. The transdermal patch of claim 1, wherein the compound formulation includes a mixture of different compounds.

14. The transdermal patch of claim 1, wherein the adhesive layer is clear to make it visually unapparent when the substrate is mounted to the skin.

15. The transdermal patch of claim 1, wherein:
the transdermal patch further comprises a backing sheet; and
the substrate is removably mounted to the backing sheet.

16. The transdermal patch of claim 15, wherein the adhesive layer is formulated to removably adhere the substrate to the backing sheet and to the skin.

17. The transdermal patch of claim 15, wherein:
the transdermal patch removably mounted to the backing sheet defines a transdermal patch sheet;
the transdermal patch defines a first transdermal patch; and
the transdermal patch sheet includes multiple transdermal patches configured the same as the first transdermal patch.

18. The transdermal patch of claim 17, wherein at least one of the transdermal patches on the transdermal patch sheet has a different shape than the first transdermal patch.

19. The transdermal patch of claim 17, wherein each transdermal patch in the transdermal patch sheet has a different ornamental design.

20. The transdermal patch of claim 19, wherein the different ornamental designs of the transdermal patches in the transdermal patch sheet are related to each by having a common theme.
